(19) United States
(12) Patent Application Publication
(10) Pub. No.: US 2026/0248553 A1
(43) Pub. Date: Aug. 27, 2026

(54) ELECTROSURGICAL IMPLEMENT

(71) Applicant: GForce Concepts Limited, Cheltenham, Gloucestershire (GB)

(72) Inventor: Gerard Michael Brooke, Cheltenham, Gloucestershire (GB)

(21) Appl. No.: 18/854,768

(22) PCT Filed: Apr. 5, 2023
(86) PCT No.: PCT/GB2023/050903

§ 371 Date: Oct. 7, 2024

(30) Foreign Application Priority Data
Apr. 8, 2022 (GB) 2205211.2

(51) Int. Cl. A61B 18/14; A61B 18/00; A61B 90/00

(52) U.S. Cl. CPC A61B 18/1442; A61B 90/03; A61B 2018/00077; A61B 2018/00083; A61B 2018/00178; A61B 2018/00184; A61B 2090/034; A61B 2218/006

(57) ABSTRACT

An electrosurgical implement (10; 110) is provided comprising a first arm (12a; 112a) having a first rigid body (30a; 130a) and a first conductive element (44a; 144a) including a first electrosurgical tip (26a; 126a) extending from a first axial tip end (24a; 124a) of the first rigid body (30a; 130a), the first rigid body (30a; 130a) comprising a first inner portion (36a; 136a) and a first outer portion (38a; 138a) connectable about the first conductive element (44a; 144a). There is also a corresponding second arm (12b; 112b) which is preferably identical to the first arm (12a; 112a). The second arm (12b; 112b) is movably connected to the first arm (12a; 112a) and there is also a biasing means for providing a separating force between the first and second arms (12a, 12b; 112a, 112b).

The present invention relates to an electrosurgical implement and a method for assembling an electrosurgical implement.

Electrosurgical implements are used widely in medical surgery for the cauterisation of wounds. An alternating current is conducted through the electrosurgical implement to the electrosurgical conductor tips, said electric current being applied to the target tissue to heat and therefore seal the tissue to prevent bleeding, amongst other things.

It is particularly important for the doctor or nurse, referenced hereon as a surgeon, using the electrosurgical implement to experience the correct ‘feel’ for the tissue that they are cauterising; otherwise, the surgeon may apply too much or too little pressure which may result in the cauterisation of excess tissue or an insufficient amount of cauterisation respectively.

In the art, standard forceps for grasping or holding target tissue have two metal arm portions through which current can be passed which are connected at a junction to form a flexible hinge. This flexible hinge does not often provide the surgeon with the correct force displacement—the correct ‘feel’—for the contact of the target tissue. The location of a hinge at a proximal portion relative to the tips also promotes the issue of the skew of the arms of the forceps, that is, where the tips do not meet one another neatly during a tweezing action.

More complex designs of electrosurgical forceps are known, but these tend to worsen the problem of ‘feel’for the surgeon and are also difficult to manufacture and sterilise.

The object of the present invention is to provide an electrosurgical implement that solves or alleviates one or more of the above stated problems.

According to a first aspect of the invention, there is provided an electrosurgical implement comprising: a first arm having a first rigid body and a first conductive element including a first electrosurgical tip extending from a first axial tip end of the first rigid body, the first rigid body comprising a first inner portion and a first outer portion connectable about the first conductive element; a second arm having a second rigid body and a second conductive element including a second electrosurgical tip extending from a second axial tip end of the second rigid body, the second rigid body comprising a second inner portion and a second outer portion connectable about the second conductive element, the second arm being movably connected to the first arm; and a biasing means for providing a separating force between the first and second arms.

This is advantageous as it is clear that the electrosurgical implement can be assembled modularly. Modular assembly promotes ease of manufacturing as features of the electrosurgical implement can be manufactured quickly and easily if one feature of the electrosurgical implement needs to be replaced.

The biasing means providing a separating force is also advantageous because this provides the electrosurgical implement with the correct force displacement for the surgeon to perform an accurate pinching motion during surgery.

Optionally, the first and second rigid bodies may preferably be made of an insulating material. Having the first and second rigid bodies being made of an insulating material protects the user from the current conducting through the implement.

Preferably, the biasing means may comprise a spring member. Furthermore, the spring member may preferably be a torsion spring. Optionally, the spring member may preferably comprise at least one sprung hinge between the first and second arms. Having a biasing means is advantageous because an effective sprung hinge portion is created, providing the correct force displacement for the surgeon to perform an accurate tweezing motion during surgery.

Advantageously, the first and second rigid bodies of the first and second arms may preferably have identical shapes to one another. This is advantageous as the first arm can be identified as the second arm and the second arm can be identified as the first arm. Therefore, the identical nature of the first and second arms promotes the ease of assembly of the electrosurgical device as the features of the first and second arms do not adversely interfere with one another. Furthermore, manufacture is greatly simplified if both arms use the same template or mould.

Preferably, each of the first and second rigid bodies of the first and second arms may have an asymmetric profile in a tweezing plane of the electrosurgical implement. Having an asymmetric profile in the tweezing plane promotes efficient interengagement of the first and second arms as the respective features of the first and second arms are spaced appropriately relative to the tweezing plane.

Optionally, the first conductive element may preferably be located between the first inner portion and the first outer portion and wherein the second conductive element may be located between the second inner portion and the second outer portion. This sandwich construction provides an interference means of holding the conductive element in position, greatly improving the speed and ease with which assembly is performed.

Preferably, the first inner portion may comprise a first electrosurgical tip support at the first axial tip end of the first rigid body to receive the first electrosurgical tip and wherein the second inner portion may comprise a second electrosurgical tip support at the second axial tip end of the second rigid body to receive the second electrosurgical tip. Having a first and second electrosurgical tip support holds the first and second electrosurgical tips in place, which prevent lateral movement of the first and second conductive elements and therefore increases accuracy of use of the electrosurgical implement.

Optionally, the first inner portion and/or the first outer portion of the first arm may preferably include a locator for receivably engaging with the first conductive element, and the second inner portion and/or the second outer portion of the second arm may preferably include a locator for receivably engaging with the second conductive element. This is advantageous as it prevents the first and second conductive elements from moving out of their position as the first and second conductive elements are secured to the respective inner portions, and is simple to assemble.

Preferably, the first rigid body may comprise at least one first retaining member and the second rigid body may comprise at least one second retaining member, the at least one first retaining member preferably being interengagable with the at least one second retaining member to inhibit displacement of first and second electrosurgical tips out of a tweezing plane of the electrosurgical implement. The interengagement of the first and second retaining members prevents the first and second arms of the electrosurgical implement from moving out of the tweezing plane and thus the first and second retaining members provide the surgeon with accurate pinching motions in surgery.

Optionally, a plurality of said first retaining members and/or a plurality of said second retaining members may be provided, the said first and second retaining members preferably interleaving with one another to inhibit displacement of first and second electrosurgical tips out of a tweezing plane of the electrosurgical implement. The availability of a plurality of first and second retaining members further prevents the displacement of the first and second arms out of the tweezing plane and therefore provides further accuracy for the surgeon when the electrosurgical implement is in use.

Preferably, the electrosurgical implement may further comprise a smoke evacuation tube positioned between the first and second arms. Having the smoke evacuation tube situated between the first and second arms provides the surgeon with a convenient placement of the smoke evacuation tube so the surgeon does not have to hold a further implement to remove the smoke produced in the cauterisation procedure. Providing a smoke evacuation tube allows the surgeon to remove smoke generated from cauterisation of a target wound to be evacuated from the surgical site. The removal of the smoke in turn provides the surgeon with a clearer view of the surgical site and also removes any unwanted odours.

Optionally, the first rigid body may preferably comprise two first retaining members and the second rigid body may comprise two second retaining members, the two first retaining members and two second retaining members preferably being interengagable with the smoke evacuation tube to inhibit displacement of first and second electrosurgical tips out of a tweezing plane of the electrosurgical implement. The placement of the smoke evacuation tube between the two first retaining members and two second retaining members results in the smoke evacuation tube being partially housed by the two first and two second retaining members and first and second rigid bodies. The placement of the smoke evacuation tube prevents the first and second arms from moving out of the tweezing plane.

The electrosurgical implement may preferably further comprise a switch associated with at least one of the first and second arms for activating a current applied to the first and second electrosurgical tips. The switch provides the user with an easy means of activating the current applied to the electrosurgical implement.

Optionally, the electrosurgical implement may preferably further comprise a conductor carrying portion engagable with the first and second arms. The conductor carrying portion can be provided as a fixed reference point relative to the user, and thus the conductor itself does not need to move with the first and second arms at the hinge portion.

Optionally, at least one of the first and second arms may preferably be movable relative to the conductor carrying portion, and wherein the conductor carrying portion may include at least one stop abuttably engagable with the first and/or second arm to limit movement of the first and/or second arm by the biasing means. This is advantageous because the conductor carrying portion prevents the hinge portion and thus the first and second arm from overextending due to the abutment of the conductor portion against the first and second arms.

Preferably, the electrosurgical implement may further comprise an electrically conductive wire, or similar conductor such as a flexible printed circuit, supported by the conductor carrying portion for providing a current to the first and second electrically conductive tips from an external power supply. Having an electrically conductive wire provides the electrosurgical implement with an electrical current to stimulate the cauterisation of a wound via the first and second electrosurgical tips.

Optionally, the electrically conductive wire may preferably comprise a first wire branch and a second wire branch, the first and second wire branches respectively connecting to the first and second conductive elements. The provision of a modular electrosurgical implement, in which the first and second arms are joined together during assembly, may require such a branched configuration. The provision of the first and second wire branch promotes the electric current to be conducted by the respective first and second conductive elements and therefore the electric current is conducted by respective first and second electrosurgical tips.

Optionally, the first arm may preferably comprise a first hinge barrel, and the second arm may comprise a second hinge barrel. This is advantageous because having the first and second hinge barrels promotes the rotational movement of the hinge as the first and second arms are moved towards and away from each other.

Preferably, the first hinge barrel may be integrally formed with the or each first retaining member, and the second hinge barrel may be integrally formed with the or each second retaining member. This provides additional stabilisation to the retaining members, limiting the propensity to flex out of the tweezing plane which could lead to lateral displacement.

According to a second aspect of the invention, there is provided a method for modular assembly of an electrosurgical implement in accordance with the first aspect of the invention, the method comprising the steps of: a] attaching the first conductive element to the first inner portion of the first arm, and attaching the second conductive element to the second inner portion of the second arm; b] making an electrical connection to the first conductive element and making an electrical connection to the second conductive element for supplying an in-use current to the first and second electrosurgical tips respectively; c] connecting the first outer portion to the first inner portion and the second outer portion to the second inner portion to respectively enclose the first and second conductive elements within the first and second rigid bodies of the first and second arms; and d] interconnecting the first and second arms with the biasing means.

Modular assembly of the electrosurgical implement is highly advantageous. The features of the electrosurgical implement that are attached to one another fit complementarily which limits the lateral movement of the electrosurgical implement.

The method identifies the ease of modular assembly for the electrosurgical implement, reducing the manufacture time. Additionally, if one of the features of the electrosurgical implement is damaged, it is straightforward to attain a feature which is identical to the damaged version and straightforward to re-assemble the electrosurgical implement.

The method for modular assembly of an electrosurgical implement may preferably further comprise a step preceding step c] of preferably providing a conductor carrying portion between the first and second inner portions. Providing a conductor carrying portion between the first and second inner portions limits the first and second arms from overextending.

The method for modular assembly of an electrosurgical implement wherein the biasing means may preferably be at least one spring member. Spring member advantageously provides the correct force displacement for the surgeon to perform an accurate pinching motion in surgery.

The method for modular assembly of an electrosurgical implement wherein the spring member may preferably be at least one torsion spring. Torsion spring advantageously provides the correct force displacement for the surgeon to perform an accurate pinching motion in surgery.

The method for modular assembly of an electrosurgical implement may preferably further comprise a step e] subsequent to step d] of placing a smoke evacuation tube between two first retaining members and two second retaining members. The provision of a smoke evacuation tube provides the user with the option to remove unwanted odours and smoke from the surgical site.

According to a third aspect of the invention there is provided an electrosurgical implement comprising: a first arm having a first rigid body housing a first conductive element, the first conductive element having a first electrosurgical tip extending from a first axial tip end thereof; a second arm having a second rigid body housing a second conductive element, the second conductive element having a second electrosurgical tip extending from a second axial tip end thereof; and a torsion spring to provide a separating force between the first and second arms.

The provision of a torsion spring providing a separating force is advantageous because the separating force creates an effective hinge portion with the correct force displacement for the surgeon to perform an accurate pinching motion when the electrosurgical implement is in use. The state of the art is biased away from the use of torsion springs, or similar rotational sprung members, since flexion of metal arms has been deemed sufficient to provide the correct ‘feel’ for a surgeon. However, the present invention provides a device which is simple to assemble by the use of the sandwich construction of the conductive elements within the first and second arms, whilst the torsion spring then provides the expected operation of the device.

According to a fourth aspect of the invention there is provided an electrosurgical implement comprising: a first arm having a first rigid body and a first conductive element including a first electrosurgical tip extending from a first axial tip end of the first rigid body, the first rigid body having a first retaining member thereon; a second arm having a second rigid body and a second conductive element including a second electrosurgical tip extending from a second axial tip end of the second rigid body, the second rigid body having a second retaining member thereon; and a hinge portion interconnecting the first and second arms at respective first and second axial hinge ends distal to the first and second axial tip ends of the first and second arms, the hinge portion having a torsion spring to provide a separating force between the first and second arms; the first and second retaining members being co-operable to limit lateral displacement of the first and second arms upon application of a user force to perform a tweezing action.

An electrosurgical implement having a torsion spring to provide the outward bias to the arms produces the expected ‘feel’ of the device, similar to that produced by very basic tweezers. However, since the torsion spring acts solely in the tweezing plane, to prevent skew of the arms relative to one another, it is preferred to add retaining members which inhibit displacement in a closed condition of the device.

The first and second arms having rigid bodies is advantageous because the rigid bodies house the internal elements of the electrosurgical implement, including but not limited to the first and second conductive elements. The rigid bodies prevent the internal elements from being damaged and also provide the surgeon with a rigid electrically-insulated grip to perform efficient surgery.

Preferably, the first and second wire branches may be respectively housed inside the first and second rigid bodies. Full enclosure can be achieved easily for the modular type of construction previously outlined.

Optionally, the first rigid body may further comprise a plurality of wire guide elements between the first inner portion and the first outer portion and wherein the second rigid body may preferably further comprise the plurality of wire guide elements between the second inner portion and the second outer portion. This is advantageous because the wire guide elements prevent the first and second wire branches from moving out of their position, reducing the likelihood of contact failure.

Preferably, the first rigid body may further comprise a first further retaining member extending from the first inner portion towards the second inner portion of the second arm to contact the second arm when the implement is in use and/or wherein the second rigid body may further comprise a second further retaining member extending from the second inner portion towards the first inner portion of the first arm to contact the first arm when the implement is in use. Having a first further retaining member is advantageous because when the first arm moves towards the second arm, the first further retaining member contacts the second arm which prevents the first and second arm from being laterally displaced. Additionally, having a second further retaining member is advantageous because when the first arm moves towards the second arm, the second further retaining member contacts the first arm which prevents the first and second arm from being laterally displaced. Having at least three co-operating retaining members inhibits lateral displacement in a layered structure, though it will be appreciated that, where the first and second arms are formed to be identical, at least four said members would be present as a minimum to achieve such inhibitive overlap.

Advantageously, the first and second hinge barrels may be respectively integrally formed with the first and second arm. The hinge portion being integrated with the first and second arms reduces the number of individual parts within the electrosurgical implement, further reducing the lateral movement of the first and second arms.

Optionally, the first and second hinge barrels may preferably be interengageable to connect the first and second arms to form the electrosurgical implement. The interengagement of the first and second hinge barrels is advantageous because this arrangement allows for the connection of the first and second arms to form the electrosurgical implement and also consequently forms the hinge portion.

Preferably, the hinge portion may further comprise a first spring receiving aperture and a second spring receiving aperture on the first and second arms respectively. Said apertures provide a suitable direct anchoring onto the first and second arms, so that the spring member does not become displaced during use.

Preferably, the first and second spring receiving apertures may respectively receive a first end portion and a second end portion of the torsion spring at the hinge portion. Having the first and second spring receiving apertures receiving the first and second end portions of the spring member means the spring member is held in place when then hinge is flexed.

Optionally, the hinge portion may preferably further comprise a holding structure and/or wherein the holding structure may preferably further comprise at least one cap, a hinge wall tube, and a tension pin to hold the first and second arms together. Having the holding structure retains the relative alignment of the hinge barrels, and provides a smooth articulating surface for the hinge.

Preferably, the first and second arms may be identical in shape to one another. Promotes ease of manufacture as the arms can be made from the same mould. Additionally, the features of each arm do not adversely interfere with one another.

Optionally, the electrically conductive wire may comprise a first and second wire branch which is respectively attached to the first and second conductive elements. Having a branching wire being specifically attached to a conductive element provides the conductive element with an electrical current.

Optionally, the branching wire may be receivable by a plurality of wire guide elements when the branching wire is connected to the conductive element. This is advantageous because the wire guide elements prevent the branching wire from moving out of its position relative to the conductive element.

The invention will now be more particularly described, by way of example only, with reference to the accompanying drawings, in which:

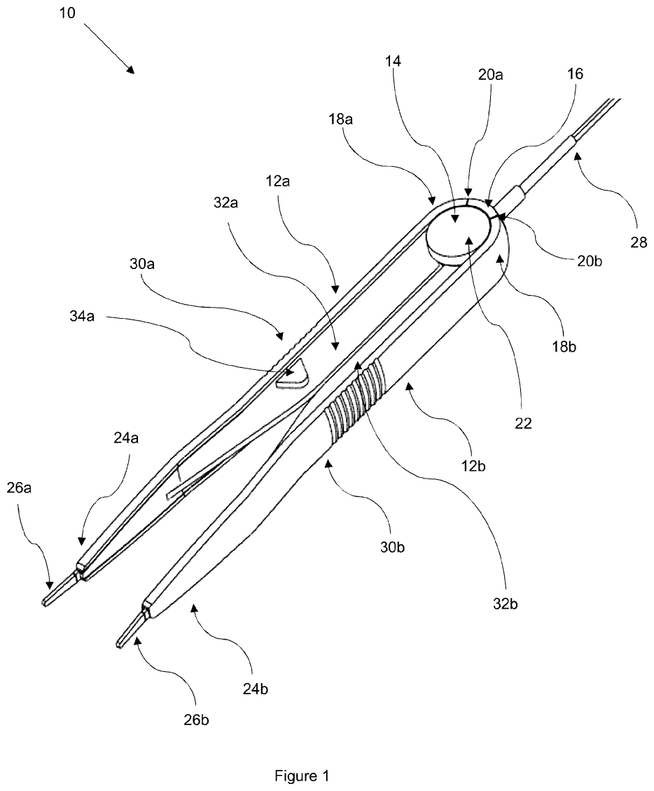

FIG. 1 shows a perspective representation of a first embodiment of an electrosurgical implement in accordance with a fourth aspect of the invention, the electrosurgical implement being in an open arrangement;

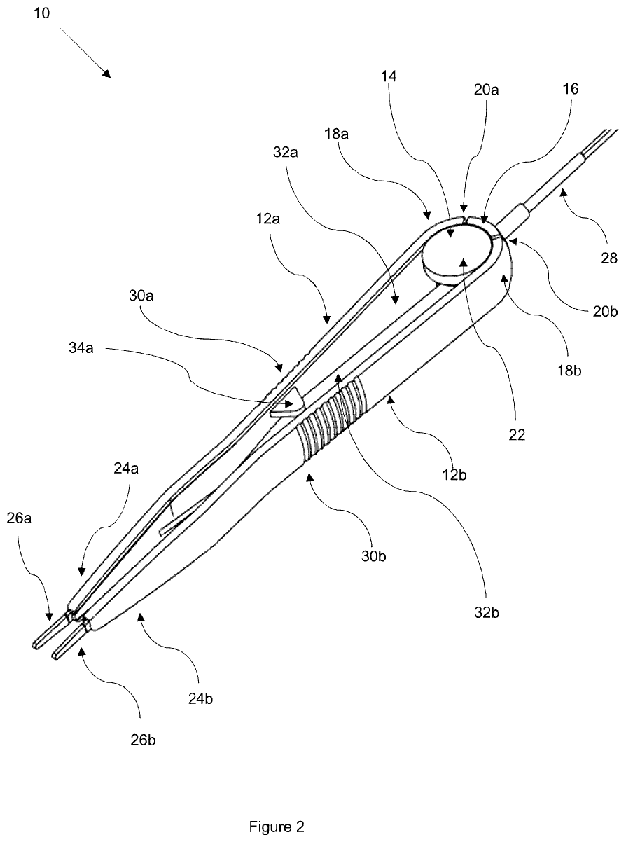

FIG. 2 shows a perspective representation of FIG. 1, the electrosurgical arrangement being in a closed arrangement;

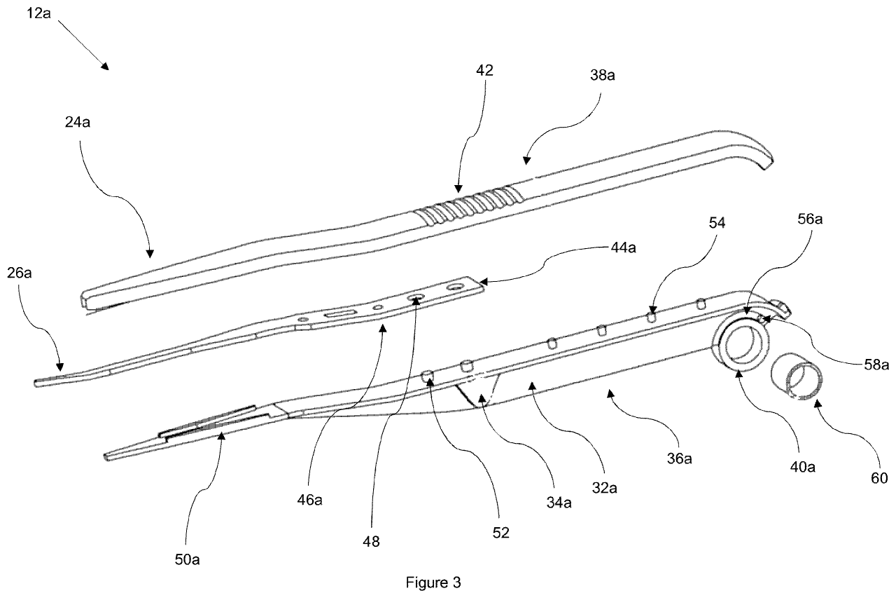

FIG. 3 shows a perspective exploded representation of a first arm of the electrosurgical implement in FIG. 1, the first arm of the electrosurgical implement being in an exploded view;

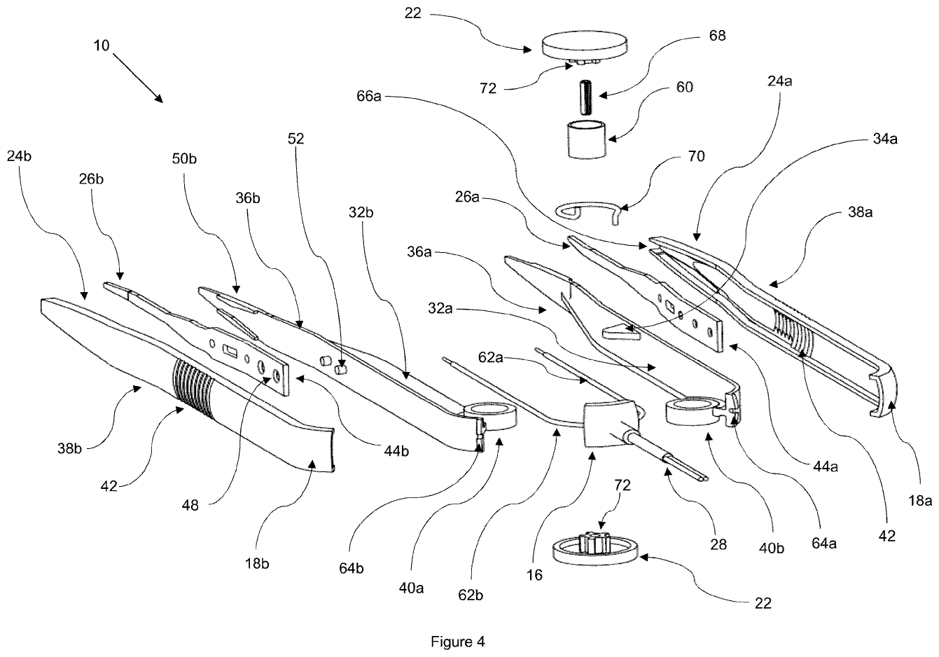

FIG. 4 shows a perspective exploded representation of the electrosurgical implement in FIG. 1;

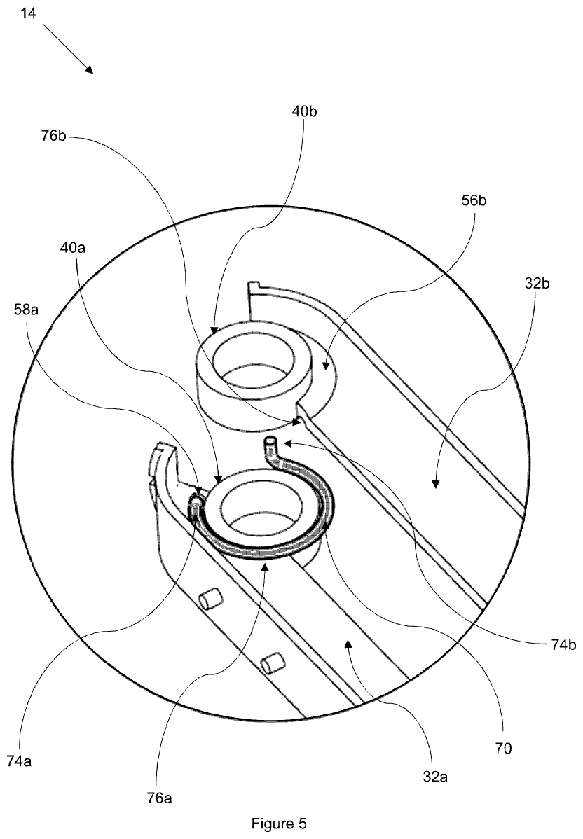

FIG. 5 shows a detailed perspective representation of a hinge portion of the electrosurgical implement of FIG. 1, specifically showing a hinge portion with a first end portion of a torsion spring being aligned with a first spring receiving aperture;

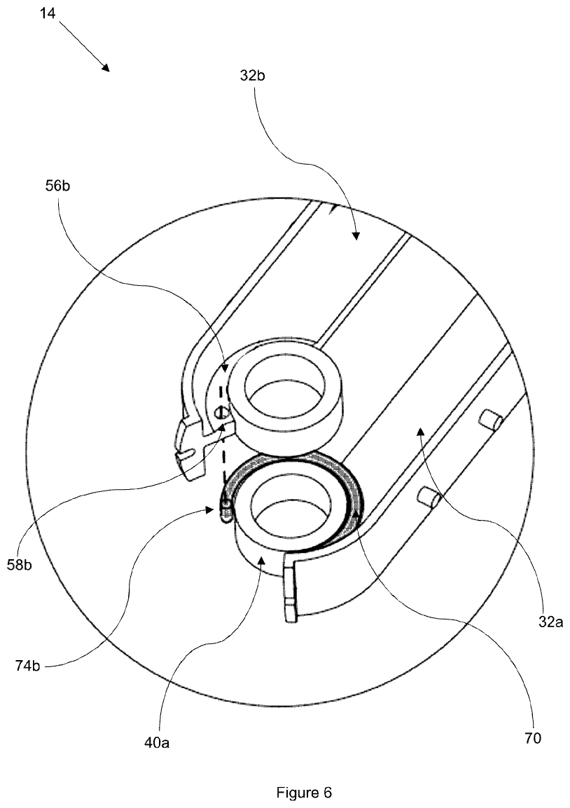

FIG. 6 shows a detailed perspective representation of a hinge portion of the electrosurgical implement of FIG. 1, specifically showing the hinge portion indicating a second end portion of the torsion spring aligned to a second spring receiving aperture.

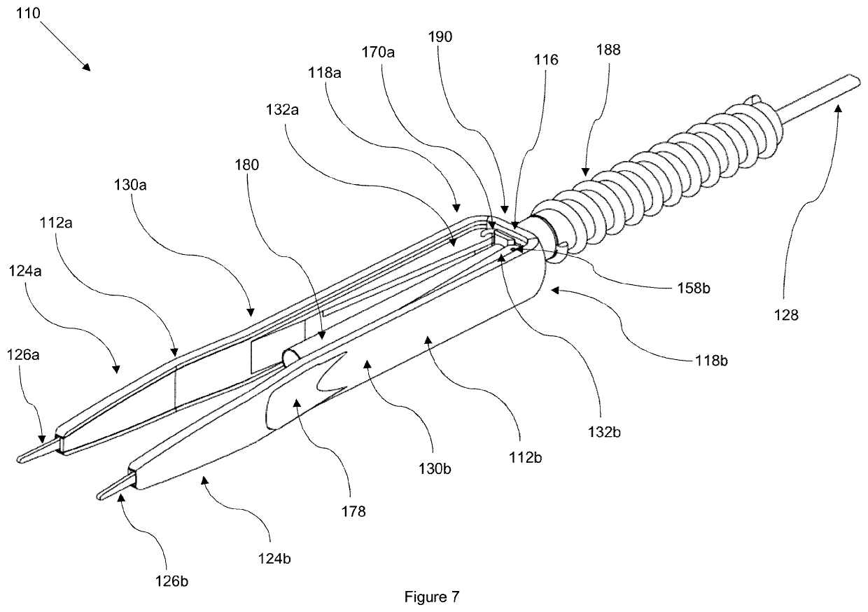

FIG. 7 shows a perspective representation of a second embodiment of an electrosurgical implement in accordance with a first aspect of the invention, the electrosurgical implement being in an open arrangement; and

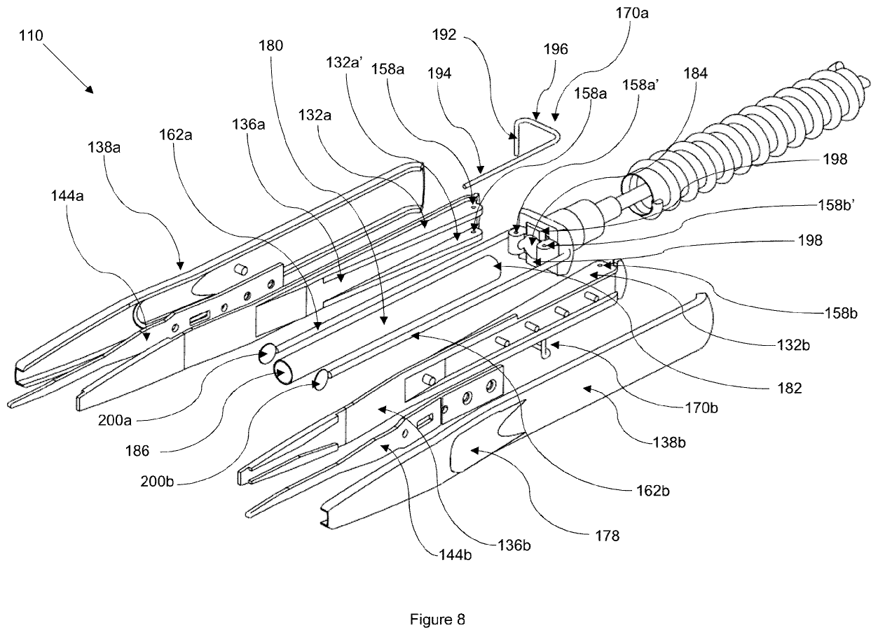

FIG. 8 shows a perspective representation of the electrosurgical implement shown in FIG. 7, the electrosurgical implement being in an exploded view.

Referring firstly to FIG. 1, there is shown an electrosurgical implement in an open arrangement, said electrosurgical implement being referenced globally at 10.

The electrosurgical implement 10 is designed to act as electrosurgical tweezers or forceps, having first and second arms 12a, 12b which are movable relative to one another via a tweezing action. This is achieved by connecting the first and second arms 12a, 12b together at a hinge portion 14. The closed arrangement of the electrosurgical implement 10 is shown in FIG. 2, in which the first and second arms 12a, 12b have been pivoted about the hinge portion 14.

The hinge portion 14 is therefore positioned at or adjacent to one end of the electrosurgical implement 10, which is an in-use user proximal end. A conductor carrying portion 16 is positioned at said end of the electrosurgical implement 10. The conductor carrying portion 16 is therefore physically positioned between respective first and second axial hinge ends 18a, 18b of the first and second arms 12a, 12b, having first and second stops 20a, 20b on either side thereof which abuttably engage with the first and second arms 12a, 12b. A cap 22, specifically an upper cap 22, is shown in FIG. 1 at the hinge portion 14, obscuring the internal workings thereof. This, and a corresponding lower cap 22, can be seen in detail in FIG. 4, however.

At an opposite end of the first and second arms 12a, 12b, hereafter first and second axial tip ends 24a, 24b respectively, are provided first and second electrosurgical tips 26a, 26b, via which current can be applied to a surgical site in use. A corresponding electrically conductive wire 28 is provided which couples the first and second electrosurgical tips 26a, 26b to an external power supply (not shown). The electrically conductive wire 28 here enters the electrosurgical implement 10 via the wire carrying portion 16, thereby forming a static reference point for the electrosurgical implement 10 about which a tweezing action can occur. Of course, an alternative conductor, such as a flexible printed circuit, could be used in place of a wire.

The electrosurgical implement 10 can be considered to have an implement axis through the centre thereof. A hinge axis of rotation or operation, which is perpendicular to the implement axis, is thus located through the hinge portion 14. A tweezing plane can therefore be defined as a plane in which the tweezing action occurs; that is, perpendicular to the hinge axis of rotation and containing the implement axis.

The first arm 12a has a first rigid body 30a and the second arm 12b has a second rigid body 30b, both of which are preferably made of plastic or another electrically insulating material. It is preferred that the first and second rigid bodies 30a, 30b are identical to one another, for ease of manufacture of the electrosurgical implement 10.

The electrosurgical implement 10 also comprises a retaining means for inhibiting lateral displacement of the arms out of the tweezing plane in use. This retaining means is here provided as co-operating retaining members respectively positioned on opposing facing surfaces of the first and second arms 12a, 12b. A first retaining member 32a is provided on the first arm 12a, which is provided as an elongate planar member extending in parallel to the tweezing plane, and a corresponding second retaining member 32b is provided on the second arm 12b, also provided as an elongate planar member extending in parallel to the tweezing plane. The first and second retaining members 32a, 32b are offset to one another with respect to the tweezing plane so as to be co-operable, rather than colliding with one another upon application of a user force to provide the tweezing action.

A first further retaining member 34a is shown on the first arm 12a in FIGS. 1 and 2, though a corresponding second further retaining member 34b is provided on the second arm 12 b. The first and/or second further retaining members 34 a, 34b provide additional lateral support to prevent displacement or skew of the first and second arms 12a, 12b out of the tweezing plane, and are further offset out of the tweezing plane. The first and second further retaining members 34 a, 34b are here provided as triangular retaining members, though it will be appreciated that any shape will be feasible, provided that a lateral retaining force is provided. In any event, the first and second further retaining members 34 a, 34b primarily provide a stabilising effect to the co-operation between the first and second said retaining members 32a, 32b, as shown in FIG. 2, and therefore can be provided to be smaller than the first and second said retaining members 32a, 32b.

FIG. 3 shows more detail regarding the arms of the electrosurgical implement 10, and is indicative of the structure of either the first arm 12a or the second arm 12b; indeed, both arms can be seen in exploded format in FIG. 4. The first arm 12a comprises a first inner portion 36a and a first outer portion 38a. As the first arm 12a and the second arm 12b are identical in shape, the second arm 12b comprises a second inner portion 36b and a second outer portion 38b. The first and second inner and outer portions 36a, 36b, 38a, 38b are preferably made of plastic or another electrically insulating material, since they combine to form the respective first and second rigid bodies 30a, 30b.

FIG. 3 will be described as being representative of the first arm 12a, though could equally be the second arm 12b, as noted above. The first inner portion 36a is shown to comprise the first retaining member 32a and the first further retaining member 34a. Similarly, the second inner portion 36b comprises the second retaining member 32b and the second further retaining member 34

b.

The first retaining member 32a is an elongate planar member which extends from a first hinge barrel 40a. This provides structural support for the first retaining member 32a at or adjacent to the hinge portion 14. The first retaining member 32a tapers as it approaches the first axial tip end 24a, to prevent impingement upon the second arm 12b during a tweezing action. Correspondingly, the second retaining member 32b of the second arm 12b is an elongate planar member which extends from a second hinge barrel 40b.

The first retaining member 32a may be integrally formed with the first inner portion 36a and extends from the first inner portion 36a.

The first further retaining member 34a also extends from the first inner portion 36a of the first arm 12a. The first further retaining member 34a is shown as having a substantially triangular cross section in a plane parallel to the tweezing plane.

The first and second outer portions 38a, 38b are each shown to comprise a grip portion 42 for the user to hold the implement. The grip portion 42 may have an undulating surface or a similar ergonomic grip suitable for providing friction to a user's fingers in use.

The first electrosurgical tip 26a of the first arm 12a is formed as part of a first conductive element 44a, which is located between the first inner portion 36a and the first outer portion 38a and is made of a conducting material such as metal. The second electrosurgical tip 26b of the second arm 12b is formed by a corresponding second conductive element 44b. The first conductive element 44a defines the first electrosurgical tip 26a, and has a first conductive body 46a which is housed inside the first rigid body 30a once the first arm 12a is assembled. The first electrosurgical tip 26a extends from the first rigid body 30a and is the feature of the electrosurgical implement 10 which creates the cauterising action.

Correspondingly, the second conductive element 44b defines the second electrosurgical tip 26b, and has a second conductive body 46b which is housed inside the second rigid body 30b once the second arm 12b is assembled.

The first conductive body 46a comprises a singular or a plurality of connection apertures 48 to attach the first conductive element 44a to the first inner portion 36a.

The first inner portion 36a is an elongate member made of an insulating material such as plastic. The first inner portion 36a has a first electrosurgical tip support 50a for receiving the first electrosurgical tip 26a of the first conductive element 44a, and therefore the first electrosurgical tip support 50a is complementarily shaped to receive the first electrosurgical tip 26a therethrough. The first electrosurgical tip support 50a is located at the first axial tip end 24a of the first arm 12a.

Correspondingly, the second inner portion 36b has a second electrosurgical tip support 50b for receiving the second electrosurgical tip 26b of the second conductive element 44b, and therefore the second electrosurgical tip support 50b is complementarily shaped to receive the second electrosurgical tip 26b therethrough. The second electrosurgical tip support 50b is located at the second axial tip end 24b of the second arm 12b.

The first inner portion 36a comprises at least one locator 52. The locator 52 is receivable by the corresponding connection aperture 48 on the first conductive element 44a. It may be feasible for there to be a projecting locator 52 on the first conductive element 44a which are receivable in receivers on the first inner portion 36a of the first arm 12a, though this may increase the difficulty of manufacture of the first conductive element 44a.

It is noted that whilst the connection of the first conductive element 44a is made to the first inner portion 36a, there is nothing precluding assembly of the first arm 12a in which the first conductive element 44a is engaged with the first outer portion 38a, using the techniques, with the locator being attached to the first and second outer portions, outlined above.

The first inner portion 36a may also comprise a plurality of wire guide elements 54 which are made of plastic or another insulating material. Four said wire guide elements 54 are illustrated in a spaced apart relationship. The wire guide elements 54 assist with holding any connecting wire between the electrically conductive wire 28 to connect to the first conductive element 44a once assembled.

The first hinge barrel 40a is located at a first axial hinge end 18a of the first arm 12a. The first hinge barrel 40a is integrally formed with the first retaining member 32a. At a first interface 56a between the first hinge barrel 40a and the first retaining member 32a there is a first spring receiving aperture 58a. Correspondingly, at a second interface 56b between the second hinge barrel 40b and the second retaining member 32b there is a second spring receiving aperture 58b.

Each first and second hinge barrel 40a, 40b is tubular and can therefore receive a wall tube 60. The wall tube 60 acts as a fulcrum within the hinge portion 14 once assembled, and spans both the first and second hinge barrels 40a, 40b of the respective first and second arms 12a, 12b to provide a smooth internal surface against which rotation of the first and second hinge barrels 40a, 40b can occur for producing the tweezing action.

The internal arrangement of the entire electrosurgical implement 10 can be seen in FIG. 4, in which both of the first and second arms 12a, 12b are shown.

The first arm 12a is asymmetrical about the tweezing plane; one side of the tweezing plane comprises the first retaining member 32a and first hinge barrel 40a and the opposite side of the tweezing plane comprises the first further retaining member 34a. As the first and second arms 12a, 12b are identical in shape, the second arm 12b is also asymmetrical in the tweezing plane and has the same respective placement of the features as the first arm 12a, albeit with the corresponding second retaining member 32b, second further retaining member 34b, and second hinge barrel 40b being on opposite sides of the tweezing plane to the counterparts of the first arm 12a.

The electrically conductive wire 28 is attached to the electrosurgical device via the conductor carrying portion 16. The electrically conductive wire 28 separates into a first wire branch 62a and a second wire branch 62b. The first and second wire branches 62a, 62b connect respectively to the first and second conductive elements 44a, 44b via the plurality of wire guide elements 54 on each first and second inner portion 36a, 36b.

There is a first wire branch receiver 64a and a second wire branch receiver 64b for receiving the respective first and second wire branches 62a, 62b. The first and second wire branch receivers 64a, 64b in the embodiment are notches and are located at the respective first and second axial hinge ends 18a, 18b of the first and second arms 12a, 12b. Specifically, the first and second wire branch receivers 64a, 64b are located at the respective first and second axial hinge ends 18a, 18b of the first and second inner portions 36a, 36b.

The first outer portion 38a is shown in more detail in FIG. 4. The first outer portion 38a further comprises a first electrosurgical tip opening 66a at the first axial tip end 24a for the first electrosurgical tip 26a to extend from the first rigid body 30a. Although not visible, a respective second electrosurgical tip opening 66b is present on the second outer portion 38 b at the second axial tip end 24b for the second electrosurgical tip 26b to extend from the second rigid body 30b.

The hinge portion 14 comprises a holding structure. The holding structure comprises two said caps, here shown as the upper and lower caps 22, a tension pin 68, the wall tube 60, a torsion spring 70, and the first and second hinge barrels 40a, 40b.

The upper and lower caps 22 each have a circular cross section parallel to the tweezing plane and can be made of an insulating material such as plastic. A tension pin receiver 72 is located on a torsion spring facing surface of both the upper and lower cap 22.

The tension pin 68 is made of metal or another resilient material such as plastic. The tension pin 68 is attachable to the tension pin receivers 72 of the upper and lower caps 22 and is surrounded by the wall tube 60 and subsequently surrounded by the first and second hinge barrels 40a, 40b, the wall tube 60 and the torsion spring 70.

The torsion spring 70 is an elongate member, the longitudinal extent of which is complementary in shape to a partial circumference of the first and second hinge barrel 40a, 40b. The torsion spring 70 has a first end portion 74a and a second end portion 74b. The first and second end portions 74a, 74b are directed away from one another. The first spring receiving aperture 58a, although not visible in FIG. 4, is located at the first interface 56a between the first hinge barrel 40a and the first retaining member 32a and the second spring receiving aperture 58b is located at the second interface 56b between the second hinge barrel 40b and the second retaining member 32b.

FIGS. 5 and 6 show a detailed view of the hinge portion 14 of the electrosurgical implement 10, and in particular, how the torsion spring 70 is inserted to assemble the electrosurgical implement 10. FIG. 5 shows the alignment of the first and second hinge barrels 40a, 40b of the respective first and second arms 12a, 12b. The first hinge barrel 40a is below the second hinge barrel 40b, parallel to the tweezing plane.

The torsion spring 70 abuts a partial circumference of the first hinge barrel 40a. The first end portion 74a of the torsion spring 70 is received by the first spring receiving aperture 58a.

The second spring receiving aperture 58b is located at the second interface 56b between the second retaining member 32b and the second hinge barrel 40b. Although not visible, the first spring receiving aperture 58a is located at the first interface 56a between the first retaining member 32a and the first hinge barrel 40a.

A first and second receiving groove 76a, 76b is present around the circumferences of each of the first and second hinge barrels 40a, 40b. This provides a locator for the torsion spring 70, ensuring it does not deform in the implement axis when a user force is applied.

The second end portion 74b of the torsion spring 70 is shown as pointing towards the second hinge barrel 40b in preparation for being received by the second spring receiving aperture 58b on the second arm 12b.

FIG. 6 shows a second detailed view of the hinge portion 14 and the alignment of the first and second hinge barrels 40a, 40b of the respective first and second arms 12a, 12b. The first hinge barrel 40a is below the second hinge barrel 40b, parallel to the tweezing plane.

The second end portion 74b of the torsion spring 70 is received by the second spring receiving aperture 58b.

The second spring receiving aperture 58b is located at the second interface 56b between the second retaining member 32b and the second hinge barrel 40b. Although not visible, the first spring receiving aperture 58a is located at the first interface 56a between the first retaining member 32a and the first hinge barrel 40a.

To assemble an electrosurgical implement 10 the manufacturer performs the following steps. The manufacturer attaches the first conductive element 44a to the first inner portion 36a of the first arm 12a using the locator 52 of the first arm 12a, the locator 52 being integrally formed with the first inner portion 36a. Correspondingly, the second conductive element 44b is attached to the second inner portion 36b of the second arm 12b using the locator 52 of the second arm 12b, the locator 52 being integrally formed with the second inner portion 36b.

The first electrosurgical tip 26a of the first conductive element 44a may be aligned by the first electrosurgical tip support 50a, and the corresponding second electrosurgical tip 26b of the second conductive element 44b may be aligned by the second electrosurgical tip support 50b.

The conductor carrying portion 16 is placed between the first and second inner portions 36a, 36b at the respective first and second axial hinge ends 18a, 18b. An electrical connection is provided by the connection of the first and second wire branches 62a, 62b to the respective first and second conductive elements 44a, 44b. The connection may be made via simple contact or soldering of the first and second wire branches 62a, 62b to the respective first and second conductive elements 44a, 44b.

The first outer portion 38a and the second outer portion 38b is attached to the respective first and second inner portions 36a, 36b, resulting in the enclosure of the first and second conductive elements 44a, 44b within the respective first and second rigid bodies 30a, 30b.

Finally, the assembled first and second arms 12a, 12b are interconnected about the conductor carrying portion 16 with the biasing means. In this instance this is achieved by engagement of the torsion spring 70 with the first and second spring receiving apertures 58a, 58b.

Once assembled, the electrosurgical implement 10 can be used in electrosurgery. The individual using the electrosurgical implement 10, typically a surgeon, holds the electrosurgical implement 10 by the grip portions 42 on the first and second arms 12a, 12b. The surgeon switches on the electrical supply, preferably by the use of the switch but may also be via an external switch not attached to the electrosurgical implement, which is conducted into the electrosurgical device via the electrically conductive wire 28.

The surgeon can apply a force to the first and second arms 12a, 12b towards each other in a tweezing action in the tweezing plane to bring the first and second arms 12a, 12b together. This creates a pincer motion at the first and second electrosurgical tips 26a, 26b.

As the first and second arms 12a, 12b move towards each other, the first and second axial hinge ends 18a, 18b of the first and second arms 12a, 12b move away from the conductor carrying portion 16. At the same time, the first and second electrosurgical tips 26a, 26b move towards each other, thereby providing the aforementioned tweezing action.

The retaining means of the electrosurgical implement inhibits the movement of the first and second arm 12a, 12b out of the tweezing plane. This is achieved by interaction of the first and second retaining members 32a, 32b. The interaction between the first and second retaining members 32a, 32b limits the lateral displacement of the first and second arms 12a, 12b in the plane of displacement. This is significantly improved by the presence of the first and second further retaining members 34a, 34b.

The surgeon can then utilise the first and second electrosurgical tips 26a, 26b to apply a cautery current as an alternating current between the first and second electrosurgical tips 26a, 26b and therefore the current passes through a targeted tissue. The first and second rigid bodies 30a, 30b are described as being made of an insulating material such as plastic. The insulating material could for instance, be formed from a resilient rubber. However, due to safety and hygiene the rigid bodies are preferably made of plastic for ease of cleaning.

FIGS. 7 and 8 show a second embodiment of an electrosurgical implement 110. Identical or similar features to the first embodiment of the invention have been omitted for brevity. The electrosurgical implement 110 can be assembled in a modular fashion in a similar manner to the first embodiment. In particular the first and second arms 112a, 112b may have first and second rigid bodies 130a, 130b having interconnectable first and second inner and outer portions 136a, 136b, 138a, 138b which house the respective first and second conductive elements 144a, 144b.

The second embodiment of the electrosurgical implement 110 is thus similar to the first embodiment with a few key differences.

In order to activate the provision of an electrical current to the electrosurgical implement 110, a switch 178 is provided. The switch 178 is located on the first rigid body 130a and/or the second rigid body 130b of the respective first arm 112a and/or second arm 112b. In the depicted embodiment a switch 178 is present on each of the first and second arms 112a, 112b.

A smoke evacuation tube 180 is provided formed of a rigid material such as rigid plastic. An attachable end 182 of the smoke evacuation tube 180 is placed within an opening 184 of the conductor carrying portion 116 of the electrosurgical implement 110. An evacuation end portion 186 of the smoke evacuation tube 180 faces towards the first and second axial tip ends 124a, 124b of the electrosurgical implement 110.

The smoke evacuation tube 180 is locatable between the two first retaining members 132a, 132a′ and the two second retaining members 132b, 132b′, the two first and two second retaining members 132a, 132a′, 132b, 132b′ forming a partial barrier to movement around the smoke evacuation tube 180.

A hose 188 is shown to extend from the conductor carrying portion 116 of the electrosurgical implement 110, the said hose 188 housing the electrically conductive wire 128. The hose 188 is fluidly communicable with the smoke evacuation tube 180 and therefore the hose 188 removes the waste smoke away from the surgical site. The hose 188 is formed of a flexible material, for example plastic or rubber.

A sprung hinge portion 190 is present between the first and second axial hinge ends 118a, 118b of the electrosurgical implement 110. The sprung hinge portion 190 comprises two springs; a first spring 170a and a second spring 170b, which provide a separating force to give the surgeon the correct force displacement when the electrosurgical implement 110 is in use. The first spring 170a hingeably connects the first arm 112a and the conductor carrying portion 116 and the corresponding second spring 170b hingeably connects the second arm 112b and the conductor carrying portion 116.

The first and second springs 170a, 170b each comprise a first end portion 192, a second end portion 194 and a bridging portion 196 between the first and second end portions 192, 194, each portion being perpendicular to one another. The bridging portion 196 is located along a spring holder 198 of the conductor carrying portion 116 when the electrosurgical implement 110 is assembled.

The first end portion 192 specifically acts as a hinge pin, whereas the second end portion 194 provides the spring action.

The two first retaining members 132a, 132a′ and two second retaining members 132b, 132b′ are positioned on opposite facing surfaces of the respective first and second arms 112a, 112b. The two first and two second retaining members 132a, 132a′, 132b, 132b′ are elongate planar members extending in parallel to the tweezing plane and taper towards the first and second axial tip ends 124a, 124b of the electrosurgical implement 110.

The two first retaining members 132a, 132a′ and two second retaining members 132b, 132b′ are interengaged with the smoke evacuation tube 180.

The two first retaining members 132a, 132a′ each have first spring receiving apertures 158a and the two second retaining members 132b, 132b′ have corresponding second spring receiving apertures 158b. The first spring receiving apertures 158a are aligned to receive a first spring 170a and the second spring receiving 158b apertures are aligned to receive an opposing second spring 170b. The first and second springs 170a, 170b are asymmetric to one another in the tweezing plane.

The conductor carrying portion 116 is also shown to have a first and a second spring receiving aperture 158a′, 158b′, as well as two spring holders 198 to each hold the bridging portion 196 of each first and second spring 170a, 170b passing from one arm to the other arm. It is noted that when the electrosurgical implement 110 is assembled, the first spring receiving apertures 158a, 158a′ of the two first retaining members 132a, 132a′ and conductor carrying portion 116 are aligned and the second spring receiving apertures 158b, 158b′ of the second retaining members 132b, 132b′ and conductor carrying portion 116 are aligned. Following this alignment, the first end portion 192 of the first spring 170a is insertable into the first spring receiving apertures 158a, 158a′ and the first end portion 192 of the second spring 170b is insertable into the second spring receiving apertures 158b, 158b′.

The conductor carrying portion 116 comprises a first wire branch 162a and a second wire branch 162b which each have a distal portion relative to a conductor carrying portion 116. A first contact element 200a and a second contact element 200b are situated at the respective distal portions of the first and second wire branches 162a, 162b. When the switch 178 of the first and/or second rigid body 130a, 130b is pressed, the first and second contact elements 200a, 200b contact the respective first and second conductive elements 144a, 144b, providing the first and second conductive elements 144a, 144b with an electric current.

As stated above, the modular assembly of the second embodiment of the electrosurgical implement 110 is similar to that of the first embodiment. However, there are a few additional factors that can be considered, as outlined below.

As the electrosurgical implement 110 is being assembled, the first and second contact elements 200a, 200b of the first and second wire branches 162a, 162b are attached to the respective first and second conductive elements 144a, 144b via soldering or other attachment means.

The first and second inner portions 136a, 136b of the first and second arms 112a, 112b are attached to the conductor carrying portion 116: the first spring receiving apertures 158a of the two first retaining members 132a, 132a′ are aligned with the first spring receiving aperture 158a′ of the conductor carrying portion 116, and correspondingly the second spring receiving apertures 158b of the two second retaining members 132b, 132b′ are aligned with the second spring receiving aperture 158b′ of the conductor carrying portion 116.

The first end portion 192 of the first spring 170a is received by the aligned first spring receiving apertures 158a, 158a′ and the first end portion 192 of the second spring 170b is received by the aligned second spring receiving apertures 158b, 158b′. The bridging portion 196 of each first and second spring 170a, 170b is placed into the respective spring holders 198.

The second end portion 194 of the first spring 170a extends along the second retaining member 132b of the second arm 112b in a tapering direction of the second retaining member 132b. The second end portion 194 of the second spring 170b extends along the first retaining member 132a′ of the first arm 112a in a tapering direction of the first retaining member 132a′. The interengagement of the first and second springs 170a, 170b with the first and second arms 112a, 112b and the conductor carrying portion 116 results in the formation of the sprung hinge portion 190.

The smoke evacuation tube 180 may be placed between two first retaining members 132a, 132a′ and two second retaining members 132b, 132b′ after the electrosurgical implement 110 is assembled. The smoke evacuation tube 180 is placed within the opening 184 of the conductor carrying portion 116 of the electrosurgical implement 110.

The placement of the smoke evacuation tube 180 results in the two first and two second retaining members 132a, 132a′, 132b, 132b′ forming a partial barrier to movement around the smoke evacuation tube 180. The smoke evacuation tube 180 is thus interengaged with the two first and two second retaining members 132a, 132a′, 132b, 132b′ and this arrangement prevents the movement of the first and second arms 112a, 112b out of the tweezing plane

It is appreciated that in this second embodiment it is possible to have an arrangement where the hose, the smoke evacuation tube and the switch are not present. Similarly, it is possible for the second embodiment to have one of either the hose, the smoke evacuation tube, or the switch. Additionally, it is possible for the second embodiment to have two of the stated optional features: the hose, the smoke evacuation tube and/or the switch.

The first and second rigid bodies have respective first and second inner and outer portions with a respective first and second conductive element between the portions. It is appreciated that the first and second rigid bodies could be made of one body rather than multiple portions. In this instance, it may be possible to slidingly engage the conductive elements into the rigid bodies during assembly.

In the first embodiment, the first and second further retaining members are both present on the respective first and second arms. However, it is possible that only one, more than two or no further retaining members are present. The further retaining members are present due to the identical shape of the first and second arms, and also to further limit the lateral displacement of the first and second arm. Therefore, having both the first and second further retaining members is preferable.

The first and second outer portions of the first and second arms are described as having a grip portion. It is possible that this grip portion could extend along the extent of the first and second outer portions. The grip portion could also be a smooth portion, an abrasive portion or another form of discontinuous portion.

It is possible that the electrosurgical implement does not have a conductor carrying portion and instead has a continuous arm where the first and second arms are combined. It is preferred that the conductor carrying portion is present, however as this prevents the first and second arms from overextending.

The electrically conductive wire is described as being attached to the electrosurgical implement via the wire carrying portion. It is appreciated that the electrically conductive wire is attached to the electrosurgical implement via, for example, the hinge portion or where the first and second wire branches respectively connect to the first and second conductive elements via entry through apertures in the first and second outer portions of the first and second arms.

It is appreciated that the plurality of wire guide elements could also be a singular wire guide element.

The first and second wire branch receivers in the embodiment are notches although it is feasible that the first and second wire branch receivers could be apertures.

In summary, it is possible to provide an electrosurgical implement for the cauterisation of wounds where the electrosurgical implement provides a correct user ‘feel’ of the contacted tissue. The effect of the correct force displacement is due to the fixed position of the sprung hinge portion at a proximal end of the electrosurgical implement along with the separating force provided by the spring member. The surgeon will experience the expected ‘feel’ of the contacted tissue and will not need to accommodate the compression force of the first and second arms they are applying to achieve the correct ‘feel’. It is also possible to provide an electrosurgical implement with a limit to the lateral displacement of the first and second arms due to the plurality of retaining members, and thus overall improving the accuracy of the electrosurgical implement when it is in use.

Additionally, it is possible to provide an electrosurgical implement which is modular. The modular nature of the electrosurgical implement results in increased ease of assembly and thus reduces the manufacture time.

Only one arm mould is required to produce the features of the electrosurgical implement as the shape of the first and second arms, and the internal features, are identical, further increasing the ease of manufacture and ensuring that the features of the first and second arms are complementary.

It is also possible to provide an electrosurgical implement with a switch directly on the first and/or second outer portions of the first and/or second rigid bodies to activate an electric current to flow through the first and/or second conductive elements. A smoke evacuation tube can also be provided to remove unwanted odours during the surgical process.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. An electrosurgical implement comprising:
a first arm having a first rigid body and a first conductive element including a first electrosurgical tip extending from a first axial tip end of the first rigid body, the first rigid body comprising a first inner portion and a first outer portion connectable about the first conductive element;
a second arm having a second rigid body and a second conductive element including a second electrosurgical tip extending from a second axial tip end of the second rigid body, the second rigid body comprising a second inner portion and a second outer portion connectable about the second conductive element, the second arm being movably connected to the first arm, wherein the first and second arms are formed as separate components to one another; and
a biasing means for providing a separating force between the first and second arms,
wherein the first and second rigid bodies of the first and second arms have identical shapes to one another, and wherein each of the first and second rigid bodies of the first and second arms have an asymmetric profile in a tweezing plane of the electrosurgical implement.

2. The electrosurgical implement as claimed in claim 1, wherein the first and second rigid bodies are made of an insulating material.

3. The electrosurgical implement as claimed in claim 1, wherein the biasing means comprises a spring member.

4. (canceled)

5. The electrosurgical implement as claimed in claim 3, wherein the spring member comprises at least one sprung hinge between the first and second arms.

6. The electrosurgical implement as claimed in claim 1, wherein the first and second rigid bodies of the first and second arms have identical shapes to one another.

7. The electrosurgical implement as claimed in claim 1, wherein each of the first and second rigid bodies of the first and second arms have an asymmetric profile in a tweezing plane of the electrosurgical implement.

8. (canceled)

9. The electrosurgical implement as claimed in claim 1, wherein the first conductive element is located between the first inner portion and the first outer portion and wherein the second conductive element is located between the second inner portion and the second outer portion.

10. The electrosurgical implement as claimed in claim 1, wherein the first inner portion comprises a first electrosurgical tip support at the first axial tip end of the first rigid body to receive the first electrosurgical tip and wherein the second inner portion comprises a second electrosurgical tip support at the second axial tip end of the second rigid body to receive the second electrosurgical tip.

11. The electrosurgical implement as claimed in claim 1, wherein the first inner portion and/or the first outer portion of the first arm includes a locator for receivably engaging with the first conductive element, and the second inner portion and/or the second outer portion of the second arm includes a locator for receivably engaging with the second conductive element.

12. The electrosurgical implement as claimed in claim 1, wherein the first rigid body comprises at least one first retaining member and the second rigid body comprises at least one second retaining member, the at least one first retaining member being interengagable with the at least one second retaining member to inhibit displacement of first and second electrosurgical tips out of a tweezing plane of the electrosurgical implement.

13. The electrosurgical implement as claimed in claim 12, wherein a plurality of said first retaining members and/or a plurality of said second retaining members is provided, the said first and second retaining members interleaving with one another to inhibit displacement of first and second electrosurgical tips out of a tweezing plane of the electrosurgical implement.

14. The electrosurgical implement as claimed in claim 1, further comprising a smoke evacuation tube positioned between the first and second arms, wherein the first rigid body comprises two first retaining members and the second rigid body comprises two second retaining members, the two first retaining members and two second retaining members being interengagable with the smoke evacuation tube to inhibit displacement of first and second electrosurgical tips out of a tweezing plane of the electrosurgical implement.

15. (canceled)

16. The electrosurgical implement as claimed in claim 1, further comprising a switch associated with at least one of the first and second arms for activating a current applied to the first and second electrosurgical tips.

17. The electrosurgical implement as claimed in claim 1, further comprising a conductor carrying portion engagable with the first and second arms.

18. The electrosurgical implement as claimed in claim 17, wherein at least one of the first and second arms is movable relative to the conductor carrying portion, and wherein the conductor carrying portion includes at least one stop abuttably engagable with the first and/or second arm to limit movement of the first and/or second arm by the biasing means.

19. The electrosurgical implement as claimed in claim 17, further comprising an electrically conductive wire supported by the conductor carrying portion for providing a current to the first and second electrosurgical tips from an external power supply, where the electrically conductive wire comprises a first wire branch and a second wire branch, the first and second wire branches respectively connecting to the first and second conductive elements.

20. (canceled)

21. The electrosurgical implement as claimed in claim 1, wherein the first arm comprises a first hinge barrel, and the second arm comprises a second hinge barrel.

22. The electrosurgical implement as claimed in claim 21, wherein the first inner portion and/or the first outer portion of the first arm includes a locator for receivably engaging with the first conductive element, and the second inner portion and/or the second outer portion of the second arm includes a locator for receivably engaging with the second conductive element, and wherein the first hinge barrel is integrally formed with the or each first retaining member, and the second hinge barrel is integrally formed with the or each second retaining member.

23. (canceled)

24. An electrosurgical implement comprising:
a first arm having a first rigid body and a first conductive element including a first electrosurgical tip extending from a first axial tip end of the first rigid body, the first rigid body comprising a first inner portion and a first outer portion connectable about the first conductive element;
a second arm having a second rigid body and a second conductive element including a second electrosurgical tip extending from a second axial tip end of the second rigid body, the second rigid body comprising a second inner portion and a second outer portion connectable about the second conductive element, the second arm being movably connected to the first arm, wherein the first and second arms are formed as separate components to one another; and
a biasing means for providing a separating force between the first and second arms,
further comprising a smoke evacuation tube positioned between the first and second arms,
wherein the first rigid body comprises two first retaining members and the second rigid body comprises two second retaining members, the two first retaining members and two second retaining members being interengagable with the smoke evacuation tube to inhibit displacement of first and second electrosurgical tips out of a tweezing plane of the electrosurgical implement.

25. An electrosurgical implement comprising:
a first arm having a first rigid body and a first conductive element including a first electrosurgical tip extending from a first axial tip end of the first rigid body, the first rigid body comprising a first inner portion and a first outer portion connectable about the first conductive element;
a second arm having a second rigid body and a second conductive element including a second electrosurgical tip extending from a second axial tip end of the second rigid body, the second rigid body comprising a second inner portion and a second outer portion connectable about the second conductive element, the second arm being movably connected to the first arm, wherein the first and second arms are formed as separate components to one another; and
a biasing means for providing a separating force between the first and second arms,
wherein the first arm comprises a first hinge barrel, and the second arm comprises a second hinge barrel.
